(19) United States
(12) Patent Application Publication
(10) Pub. No.: US 2017/0007537 A1
(43) Pub. Date: Jan. 12, 2017

(54) PHARMACEUTICAL COMPOSITIONS OF ASENAPINE

(71) Applicant: HETERO RESEARCH FOUNDATION, Hyderabad, Andhra Pradesh (IN)

(72) Inventors: BANDI PARTHASARADHI REDDY, HYDERABAD, ANDHRA PRADESH (IN); PODILI KHADGAPATHI, HYDERABAD, ANDHRA PRADESH (IN); PATCHIGOLLA SATYANARAYANA RAO, HYDERABAD, ANDHRA PRADESH (IN)

(73) Assignee: HETERO RESEARCH FOUNDATION, HYDERABAD, ANDHRA PRADESH (IN)

(21) Appl. No.: 15/119,439

(22) PCT Filed: Feb. 16, 2015
(86) PCT No.: PCT/IN2015/000089

§ 371 Date: Aug. 17, 2016

(30) Foreign Application Priority Data
Feb. 18, 2014 (IN) 753/CHE/2014

(51) Int. Cl. A61K 9/00; A61K 31/407

(52) U.S. Cl. CPC A61K 9/006; A61K 31/407

(57) ABSTRACT

The present invention relates to liquid compositions of asenapine with one or more pharmaceutically acceptable excipients. More particularly, the present invention relates to liquid spray compositions comprising asenapine for administration through oral mucosa.

### PRIORITY

This patent application claims priority to Indian patent application number 753/CHE/2014, filed on Feb. 18, 2014, the contents of which are incorporated by reference herein in their entirety.

### FIELD OF THE INVENTION

The present invention relates to pharmaceutical compositions comprising asenapine and one or more pharmaceutically acceptable excipients.

### BACKGROUND

Asenapine is a psychotropic agent belongs to the class dibenzo-oxepino pyrroles. Asenapine maleate is chemically described as (3aRS,12bRS)-5-Chloro-2-methyl-2,3,3a,12b-tetrahydro-1Hdibenzo[2,3:6,7]oxepino[4,5-c]pyrrole(2Z)-2-butenedioate (1:1) and has the following structural formula,

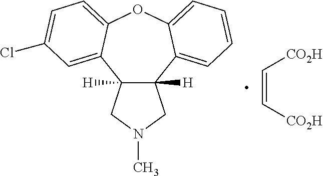

In the United States, Asenapine is available as 5 mg and 10 mg sublingual tablets with trade name Saphris® by Organon Sub Merck.

As per the literature, absolute bioavailability of asenapine when swallowed is low (<2% with an oral tablet formulation) which may be due to first pass effect.

U.S. Pat. No. 5,763,476 disclose sublingual tablet compositions of asenapine and preparation thereof by freeze drying process which is expensive and tedious. Moreover, the formulations disclosed are porous in nature which may get eroded thereby increasing the chances of being swallowed which may lead to wastage of dose. This patent also reported that per-oral administration of asenapine may result in cardiovascular adverse events.

Further, sublingual tablets have the disadvantage of increased chances of being swallowed as such involuntarily or due to unacceptable bitter taste.

US 2008/0306133 disclose intranasal dosage formulation of asenapine for absorption through nasal mucosa. Some amount of the formulation, when administered through intranasal route have the chances of getting escaped and entering into oro-pharynx and being swallowed or into respiratory tract through nasal cavity. Moreover, as of date intra nasal administration of asenapine have no clear cut safety evaluations and may be irritating to the patients using such dosage forms.

Still, there exists a need to develop alternative formulations of asenapine. Accordingly, inventors of the present invention have developed oral spray formulations of asenapine.

The present invention has the following advantages:
Increased surface area of absorption, as delivery of the drug is not restricted to sublingual route.
Increased retention of the formulation in contact with the oral mucosa for higher bioavailability.
Delivery of precise dose (enhanced availability of drug for absorption) owing to the usage of metered dispensing system.
The dosage form has increased ease of use/compliance.
Avoids the risk of swallowing as in the sublingual dosage forms, thus reducing the risk of cardiovascular adverse events also.
Increased patient acceptability.

### SUMMARY OF THE INVENTION

The present invention relates to oral spray compositions of asenapine. More particularly, the present invention relates to pharmaceutical compositions comprising asenapine for administration through buccal mucosa.

One embodiment, of the present invention relates to pharmaceutical liquid spray composition for administration through buccal mucosa comprising asenapine or a pharmaceutically acceptable salt thereof and one or more pharmaceutically acceptable excipients.

Another embodiment of the present invention relates to pharmaceutical liquid composition comprising:
(a) asenapine or a pharmaceutically acceptable salt thereof,
(b) permeation enhancer,
(c) at least a cosolvent,
(d) at least a mucoadhesive polymer and,
(e) an optional surfactant.

One another embodiment of the present invention relates to oral spray composition comprising asenapine or a pharmaceutically acceptable salt thereof, Diethylene glycol monoethyl ether and one or more other pharmaceutically acceptable excipients.

Other embodiment of the present invention relates to liquid compositions of asenapine for administration via metered dispensing system for increased bioavailability wherein, the said metered dispensing system is a spray pump and/or an actuator system with simple, user-friendly operation to deliver precise dose over buccal mucosa.

Further embodiment of the present invention relates to method of treating schizophrenia, bipolar disorder in a patient in need thereof comprising administering to the patient the composition of the present invention.

### DETAILED DESCRIPTION OF THE INVENTION

The present invention relates to oral spray compositions of asenapine. More particularly, the present invention relates to pharmaceutical compositions comprising asenapine for administration through buccal mucosa.

The term “oral” as used here in the present invention is not intended to be per-oral but is for oral route of administration intended to be absorbed through oral mucosa more particularly through buccal mucosa.

The present invention relates to a method which comprises administering via oral mucosal exposure an effective amount of asenapine or a pharmaceutically acceptable salt thereof to a patient in need thereof.

The term “active ingredient” or “active agent” or “drug” used interchangeably, is defined to mean active drug (e.g. asenapine), that can induce a desired pharmacological or physiological effect.

The term “asenapine” as used herein according to the present invention includes asenapine in the form of free base, a pharmaceutically acceptable salt thereof, amorphous asenapine, crystalline asenapine or any isomer, polymorph, derivative, hydrate, solvate, or prodrug or combinations thereof preferably, asenapine maleate in an amount of 10 mg per less than or equal to 0.5 ml of the composition.

The term “pharmaceutically acceptable” as used herein means that which is useful in preparing a pharmaceutical composition that is generally safe and non-toxic.

The term “excipient” means a pharmacologically inactive component such as a surfactant, preservative, solvent, co-solvent, carrier, permeation enhancer, muco adhesive polymer, buffer, taste modifier, flavor and the like of a pharmaceutical product. The excipients that are useful in preparing a pharmaceutical composition are generally safe, non-toxic and are acceptable for human use. Reference to an excipient includes both one and more than one such excipients.

The term “composition” or “pharmaceutical composition” as used herein synonymously include liquid dosage forms.

As used in this specification, the singular forms “a”, “an”, and “the” include plural references unless the context clearly dictates otherwise. Thus for example, a reference to “a method” or “a process” includes one or more methods, one or more processes and/or steps of the type described herein and/or which will become apparent to those persons skilled in the art upon reading this disclosure and so forth.

The term “oral mucosa” as used in the present invention can be buccal, sublingual routes of administration more particularly buccal route.

One embodiment of the present invention relates to pharmaceutical liquid spray composition for administration through buccal mucosa comprising asenapine or a pharmaceutically acceptable salt thereof and one or more pharmaceutically acceptable excipients.

The liquid pharmaceutical composition according to the present invention is in the form of solution, suspension, nano-suspension, emulsion, micro-emulsion, multiple emulsion and the like meant for administration through oral mucosa preferably, in the form of solution. The compositions further comprise delivery systems such as solid lipid nanoparticles, liposomes and the like.

Another embodiment of the present invention relates to pharmaceutical liquid composition comprising:
(a) asenapine or a pharmaceutically acceptable salt thereof,
(b) permeation enhancer,
(c) at least a cosolvent,
(d) at least a mucoadhesive polymer and,
(e) an optional surfactant.

Permeation enhancer as used in the present invention is selected from one or more of diethylene glycol monoethyl ether, poloxamers, phosphotidyl choline, bile salts, cyclodextrins, menthol and cetrimide and the like and combinations thereof in an amount of from 1 to 60% by weight, preferably from 10% to 50% by weight.

Diethylene glycol monoethyl ether as used in the present invention is marketed by Gattefosse under the brand name “Transcutol®” is synonymously referred to as Carbitol, 3,6-Dioxa-1-octanol, Diethylene glycol ethyl ether, Diglycol monoethyl ether, Dioxitol, Ethanol 2,2′-oxybis-monoethyl ether, Ethyl carbitol, Ethyl diethylene glycol, Ethyl digol.

Co-solvents as used in the present invention selected from one or more of ethanol, isopropyl alcohol, acetone, benzyl alcohol, polyhydric alcohols such as glycerine, propylene glycol and polyethylene glycols, glycol ethers and the like and combinations thereof in an amount of from 1% to 10% by weight, preferably from 3% to 8% by weight.

Polymers as used in the present invention is a mucoadhesive polymer selected from one or more of celluloses and derivatives such as hydroxypropyl methylcellulose, hydroxyethyl cellulose, hydroxypropyl cellulose, ethyl cellulose, methyl cellulose, carboxymethyl cellulose sodium, cellulose acetate phthalate and the like, natural gums such as xanthan gum, karaya gum, guar gum and the like, carbomers, chitosan, copolymers of acrylic acid such as eudragit, polyvinyl alcohol, Polyvinylpyrrolidone and the like or is an in-situ gelling agent selected from poloxamers, gellan gum, alginic acid, xyloglucan, pectin, chitosan, poly (D,L-lactic acid), poly (D,L-lactide-co-glycolide), poly-caprolactone and the like and combinations thereof in an amount of from 1% to 20% by weight preferably, from 1% to 10% by weight.

Surfactants as used in the present invention are selected from one or more of anionic surfactants such as sodium lauryl sulfate, docusate sodium, 2-Naphthalene sulfonate sodium, cationic surfactants such as cetylpyridinium chloride, benzalkonium chloride, zwitterionic surfactants such as lecithin, nonionic surfactants such as polysorbates, sorbitan esters, castor oil and its derivatives such as polyoxyl 40 hydrogenated castor oil and the like and combinations thereof in an amount of from 1% to 10% by weight.

The present invention further comprises other pharmaceutically acceptable excipients selected from one or more of preservatives, buffers, taste modifiers, flavors or combinations thereof.

Preservatives as used in the present invention selected from one or more of benzalkonium chloride, benzyl alcohol, chlorobutanol, cresol, ethyl alcohol, thiomersal, parabens, benzoic acid, sodium benzoate and the like thereof.

Buffers as used in the present invention include an acid or a base and its conjugate base or acid, respectively. Suitable buffers include mixtures of weak acids and alkali metal salts (e.g., sodium, potassium) of the weak acids, such as acetate, citrate, tartarate, phosphate, benzoate and bicarbonate buffers and combinations thereof.

Taste modifiers used in the present invention increases the patient acceptability and are selected from one or more of ion exchange resins, polyvinyl pyrrolidone and/or copolyvidone, a high molecular weight polyethylene glycol, sweetening agents such as monosaccharides, disaccharides, sugar alcohols, and polysaccharides, e.g., glucose, fructose, invert sugar, sorbitol, sucrose, maltose, xylose, ribose, mannose, corn syrup solids, xylitol, mannitol, maltodextrins, and mixtures thereof, artificial sweeteners and dipeptide-based sweeteners such as saccharin salts, acesulfame K, sucralose, aspartame, and mixtures thereof.

Flavors may optionally be used in the present invention selected from one or more of natural such as naturally derived oils from plants, flowers, leaves, nature identical, artificial flavoring compounds such as synthetic flavor oils.

The term “solvent” refers to an ingredient used for dissolving an ingredient. Suitable solvents that can be used according to the present invention includes but not limited to purified water or organic solvent or a mixture of purified water and an organic solvent selected from ethanol or tertiary-butyl alcohol or isopropyl alcohol or methanol or methylene chloride or ethyl acetate or acetone and combinations thereof.

In an another embodiment, the present invention relates to oral spray composition comprising asenapine or a pharmaceutically acceptable salt thereof, Diethylene glycol monoethyl ether and one or more other pharmaceutically acceptable excipients.

Diethylene glycol monoethyl ether in the composition according to the present invention is in an amount of from 5 to 20 parts to each part of asenapine by weight preferably, from 5 to 15 parts by weight to each part of asenapine.

Further embodiment of the present invention relates to process for the preparation of pharmaceutical composition comprising asenapine comprising the steps of:
a) dissolving permeation enhancer in part of purified water to form a solution and dispersing asenapine in the solution and stirring to form a clear solution,
b) dissolving polymer in part of purified water to form a clear solution,
c) adding cosolvent, and other desirable excipients to solution of step (a) and stirring to form a clear solution,
d) adding solution of step (b) to the solution of step (c) and stirring to form a clear solution,
e) making up the final volume using purified water and filling into a dispensing system.

In an another embodiment, the present invention relates to liquid compositions of asenapine for administration via metered dispensing system for increased bioavailability wherein, the said metered dispensing system is a spray pump and/or an actuator system with simple, user-friendly operation to deliver precise dose over buccal mucosa.

The present invention has increased retention of the formulation in contact with the oral mucosa resulting in higher bioavailability and also has enhanced ease of usage provided by a spray pump and/or an actuator system with simple, user-friendly operation to deliver precise dose of asenapine.

Further embodiment of the present invention relates to method of treating schizophrenia, bipolar disorder in a patient in need thereof comprising administering to the patient the composition of the present invention.

### EXAMPLES

The following examples further describe and demonstrate particular embodiments within the scope of the present invention. The examples are given solely for illustration and are not to be construed as limitations as many variations are possible without departing from spirit and scope of the invention.

### Example 1

### Liquid Compositions of Asenapine

| Ingredient | % w/w |
|---|---|
| Asenapine maleate | 3.50 |
| Diethylene glycol monoethyl ether | 25.00 |
| Glycerine | 7.5 |
| Hydroxypropyl methylcellulose | 1.03 |
| Benzyl alcohol | 1.00 |
| Sucralose | 0.025 |
| Cherry flavour | 0.10 |
| Purified water | q.s. |

3.50% of asenapine maleate is equivalent to 2.49% of asenapine

### Brief Manufacturing Process:

1. dissolve diethylene glycol monoethyl ether in part of purified water to form a solution and disperse asenapine in the solution and stir to form a clear solution,
2. dissolve hydroxypropyl methylcellulose in part of purified water to form a clear solution,
3. add glycerine, benzyl alcohol, sucralose and cherry flavour to solution of step 1 and stir to form a clear solution,
4. add solution of step 2 to the solution of step (3) and stir,
5. make up the final volume using purified water and fill into a dispensing system.

### Example 2-3

### Liquid Compositions of Asenapine

| | Example 2 | Example 3 |
|---|---|---|
| Ingredients | (% w/w) | (% w/w) |
| Asenapine maleate^{#} | 3.500 | 3.500 |
| Poloxyl-40 Hydrogenated Castor oil | 12.50 | 11.10 |
| Polyethylene glycol | 5.00 | 4.50 |
| Poloxamer 407* | 20.00 | 5.84 |
| Hydroxypropyl methylcellulose | — | 4.50 |
| Benzyl alcohol | 1.00 | 1.00 |
| Sucralose | 0.025 | — |
| Cherry flavour | 0.10 | — |
| Aspartame | — | 0.30 |
| Acesulfame K | — | 0.10 |
| Prosweet | — | 0.80 |
| Purified water | | q.s. |

^{#}3.50% of asenapine maleate is equivalent to 2.49% of asenapine.
*Poloxamer is polyoxyethylene-polyoxypropylene block copolymer

### Brief Manufacturing Process:

1. dissolve asenapine in part of purified water and stir to form a clear solution,
2. dissolve hydroxypropyl methylcellulose or poloxamer in part of purified water to form a clear solution,
3. add polyethylene glycol other excipients to solution of step 1 and stir to form a clear solution,
4. add solution of step 2 to the solution of step (3) and stir,
5. make up the final volume using purified water and fill into a dispensing system.

### Example 4 to 6

### Liquid Compositions of Asenapine

| | Example 4 | Example 5 | Example 6 |
|---|---|---|---|
| Ingredients | (% w/w) | (% w/w) | (% w/w) |
| Asenapine maleate^{#} | 2.0 | 2.0 | 3.0 |
| Docusate sodium | 0.5 | — | — |
| Tocopherol Polyethylene | — | 0.75 | — |
| glycol succinate | | | |
| Aspartame | 0.30 | — | — |
| Saccharine sodium | — | 0.15 | — |
| Prosweet | — | — | 1.0 |
| Sodium benzoate | 0.2 | — | — |
| Benzyl alcohol | — | 1.0 | |
| Methyl paraben | — | — | 0.2 |
| Purified water | q.s. | q.s. | q.s. |

^{#}2.0% of asenapine maleate is equivalent to 1.4% of asenapine.
^{#}3.0% of asenapine maleate is equivalent to 2.1% of asenapine.

### Example 7

### Liquid Compositions of Asenapine

| Ingredients | % w/w |
|---|---|
| Asenapine maleate^{#} | 3.515 |
| Diethylene glycol monoethyl ether | 7.50 |
| Poloxamer 407* | 20.00 |
| Benzyl alcohol | 1.00 |
| Sorbitol | 15.00 |
| Purified water | q.s. |

^{#}3.515% of asenapine maleate is equivalent to 2.50% of asenapine.
*Poloxamer is polyoxyethylene-polyoxypropylene block copolymer

### Example 8

### Liquid Compositions of Asenapine

| Ingredients | % w/w |
|---|---|
| Asenapine maleate^{#} | 3.515 |
| d-Limonene | 7.50 |
| Carbomer | 0.20 |
| Benzyl alcohol | 1.00 |
| Propylene glycol | 5.00 |
| Sucralose | 0.05 |
| Purified water | q.s. |

^{#}3.515% of asenapine maleate is equivalent to 2.50% of asenapine.

### Example 9

### Liquid Compositions of Asenapine

| Ingredients | % w/w |
|---|---|
| Asenapine maleate^{#} | 3.515 |
| Polyoxyethylated oleic glycerides | 12.50 |
| Xanthan gum | 0.20 |
| Sodium Methyl paraben | 1.00 |
| Polyethylene glycol | 10.00 |
| Purified water | q.s. |

^{#}3.515% of asenapine maleate is equivalent to 2.50% of asenapine.

### Example 10

### Liquid Compositions of Asenapine

| Ingredients | % w/w |
|---|---|
| Asenapine maleate^{#} | 3.515 |
| Polyethylene glycol | 70.00 |
| Sodium glycocholate | 2.50 |
| Starch | 2.50 |
| Benzalkonium chloride | 0.02 |
| Disodium Edetate | 0.01 |
| Prosweet flavor enhancer | 0.10 |
| Purified water | q.s. |

^{#}3.515% of asenapine maleate is equivalent to 2.50% of asenapine.

### Example 11

### Liquid Compositions of Asenapine

| Ingredients | % w/w |
|---|---|
| Asenapine maleate^{#} | 3.515 |
| Polyglycerol ester of mixed fatty acids | 7.50 |
| Sodium Stearyl fumarate | 2.50 |
| Sodium Benzoate | 0.20 |
| Propylene glycol | 10.00 |
| Aspartame | 0.01 |
| Purified water | q.s. |

^{#}3.515% of asenapine maleate is equivalent to 2.50% of asenapine.

### Example 12

### Liquid Compositions of Asenapine

| Ingredients | % w/w |
|---|---|
| Asenapine maleate^{#} | 3.515 |
| Hydroxypropyl beta cyclodextrin | 7.50 |
| Glycerine | 10.00 |
| Sodium Benzoate | 0.20 |
| Citric acid | 0.15 |
| Menthol | 0.01 |
| Purified water | q.s. |

^{#}3.515% of asenapine maleate is equivalent to 2.50% of asenapine.

### Brief Manufacturing Process (for Examples 4-12):

1. dissolve the excipients in purified water to form a solution,
2. dissolve asenapine in the solution of step 1 to form a clear solution,
3. make up the final volume using purified water and fill into a dispensing system.

1. A pharmaceutical liquid spray composition for administration through buccal mucosa comprising asenapine or a pharmaceutically acceptable salt thereof and one or more pharmaceutically acceptable excipients.

2. The composition according to claim 1, in the form of a solution, a suspension, a nano-suspension, an emulsion, a micro-emulsion, or a multiple emulsion.

3. The composition according to claim 1, wherein the asenapine is in the form of asenapine maleate.

4. A pharmaceutical liquid composition according to claim 1, comprising
a permeation enhancer,
a cosolvent,
a mucoadhesive polymer and,
optionally a surfactant.

5. The composition according to claim 4, wherein the asenapine is in the form of asenapine maleate.

6. The composition according to claim 4, wherein the permeation enhancer is diethylene glycol monoethyl ether, a poloxamer, phosphotidyl choline, a bile salt, a cyclodextrins, menthol, cetrimide, or a combination comprising one or more of the foregoing.

7. The composition according to claim 4, wherein the cosolvent is ethanol, isopropyl alcohol, acetone, benzyl alcohol, a polyhydric alcohols, or a combination comprising one or more of the foregoing.

8. The composition according to claim 4, wherein the mucoadhesive polymer is hydroxypropyl methylcellulose, hydroxyethyl cellulose, hydroxypropyl cellulose, polyvinylpyrrolidone, polyvinyl alcohol, xanthan gum, chitosan, a carbomer, maize starch, sodium stearyl fumarate, or a combination comprising one or more of the foregoing.

9. The composition according to claim 4, wherein the surfactant is polyoxyl 40 hydrogenated castor oil, sodium lauryl sulfate, docusate sodium, 2-Naphthalene sulfonate sodium, cetylpyridinium chloride, benzalkonium chloride, lecithin, polysorbates, a sorbitan ester, or a combination comprising one or more of the foregoing.

10. An oral spray composition comprising asenapine or a pharmaceutically acceptable salt thereof, diethylene glycol monoethyl ether, and one or more pharmaceutically acceptable excipients.

11. The composition according to claim 10, wherein the diethylene glycol monoethyl ether is present in an amount of from 5 to 15 parts to each part of asenapine by weight.

12. A metered dispensing system for increased bioavailability of asenapine, wherein the metered dispensing system is a spray pump and/or an actuator system to deliver a precise dose of asenapine over the buccal mucosa.

13. The metered dispensing system according to claim 12, wherein asenapine is in the form of asenapine maleate.

14. A method of treating schizophrenia, or bipolar disorder in a patient in need thereof comprising administering to the patient the composition of claim 1.
